(19) United States
(12) Patent Application Publication
(10) Pub. No.: US 2013/0213116 A1
(43) Pub. Date: Aug. 22, 2013

(54) METHOD AND DEVICE FOR LAND MINE DETECTION BY NITROGEN GAS

(71) Applicant: Ahmet Zengin, Ankara (TR)

(72) Inventor: Ahmet Zengin, Ankara (TR)

(21) Appl. No.: 13/743,577

(22) Filed: Jan. 17, 2013

(30) Foreign Application Priority Data
Jul. 26, 2010 (TR) 2010/06134

(51) Int. Cl. G01N 33/00

(52) U.S. Cl. CPC G01N 33/0057
USPC 73/31.02

(57) ABSTRACT

A land mine detection device that includes an air analysis sensor which measures the air in the environment in which it is located and determines the amount of inorganic nitrogen found in the environment, and a processor unit which controls the system.

### TECHNICAL FIELD

The invention relates to the method and device for land mine detection by nitrogen gas method.

### BACKGROUND

A mine is an explosive designed for destroying or damaging land vehicles, ships or aircraft or for injuring, killing or otherwise neutralizing personnel and which normally is inside a protective coating material. Principally it is grouped in 2 main groups, these being land and sea. The types of mine most frequently encountered among land mines are anti-personnel and anti-tank mines. It is possible to say that in this group antitank mines are larger and more powerful as compared to anti-personnel mines.

A mine displays its activity as the result of contact and the pressure applied on it. Therefore, mine detection operations are required to be performed without contacting the mine. When today's land mine detection methods are considered, it would be possible that differences methods are used and that these methods have differences advantages as compared to one another. Detection of land mines with the aid of metal detectors is encountered as a method which is frequently used. In scanning for mines with the aid of metal detectors, the detection of metals buried under the soil is made. However, due to the fact that this method remains powerless especially against plastic land mines and it does not differentiate whether the metal detected underground is a mine, it would be possible to say that its efficiency is low. Thermal neutron activation detectors, even though increase the success of the detection method by making this differentiation, the fact that this device is a slow, costly and large detector for use on the field, reduces its effectiveness.

Among electromagnetic detection systems, soil penetration radar systems take the most important place. This system produces radio waves undertake the soil and measures the frequency of the signals received. As the result of this measurement, objects present under the ground the detected and the mine finding operation is thus performed. The strongest aspect of this detection method is that it is more successful in comparison to metal detectors in prevalent use; because this system captures variations under the soil better and is used for the detection of mines which are not made from metal. At the same time, this method is a known and mature technology which is easy to apply. However, this detection method also has certain disadvantages. Namely, large rocks, water bodies or large tree roots found under the soil may result in a distortion of the waves. As the result of this, erroneous signals are received and the success of the system is reduced.

Its second greatest disadvantage is that the depth to which the detector is inserted in the soil should not be too much. In this case the detection of mines which are hidden somewhat deeply cannot be made.

Another one of innovative mine detection methods is the method of tracing with x-ray device. In this mine detection method which is of a nature parallel to x-ray technology, it is attempted to take a photograph of the underground via the rays sent into the soil. The images obtained by this means are interpreted for the existence of a mine. The strong aspect of the system is that it can fully reflect the physical location and view of the mine. However, problems such as the fact that it can detect mines which are at most 10 cm deep under the ground, the long time required for it to be able to detect at a greater depth, and the device's sensitivity to vibrations encountered during the scan may be listed as the major shortcomings of the system.

It is known that infrared technologies are also used in mine detection systems. With these systems, to observe the level and variation of the electromagnetic radiation in the soil is aimed. Through this system it is possible to scan large areas in a short time. At the same time, there is no physical dependency on the person to conduct the scanning. Therefore, the risk is fairly low. However, the fact that the system is considerably sensitive to the environmental changes existing in the external environment necessitates the weather conditions to be considerably favorable. For example, it is fairly difficult to perform mine detection in windy, rainy or foggy weather.

Today, it is also possible to detect mines using acoustic or seismic data. On the basis of these systems is the idea of detecting the mine's location through vibrations. Objects of different nature display vibrations at different rates against the same impact. This understanding dominates the basis of the systems developed. The greatest disadvantage of this detection method, which, with its low faulty alarm rate, has a significant effectiveness among other systems, is that it cannot detect a mine which is located deeply; because it is fairly difficult for the response to be taken from a mine buried deep to reach the surface. The fact that the detection process is lengthy is one of the significant shortcomings of the method.

Apart from mechanical or electronic methods, some biological methods are also known to be used in mine detection operations. With this method, by routing mammals, insects or certain microorganisms to the region in which mines are to be detected, the reactions that the creatures display are examined and the presence of mines are sought according to their manner of behavior. In these detection methods which make use of dogs and mice among mammals the smell which occurs in the mined area forms the basis of the detection system. The greatest advantage of these methods is that detection work can be conducted under different geographical and weather conditions. Additionally, the fact that the mines can be detected without detonating, due to the weights of the creatures, is one of its important strong points. However, that the success of the method depends on the quality of training of the creatures and on their threshold of smell is a disadvantage. In addition to this, the fact that the explosive smell disperses or diminishes in bad weather conditions gives rise to the result of faulty detection. Additionally, it is possible to say that this method is not efficient in terms of time.

In the detection method which is the subject matter of the invention, on the other hand, it is a method in which the disadvantages listed for various methods are absent and which enables the detection of mines in a very short time without errors. With this method the possibility of erroneous signals, found in our detection methods is eliminated; because the content of every land mine comprises concentrated nitrogen and intensively releases nitrogen in inorganic form to the environment in which it is found. Therefore, factors such as bad weather conditions or the structure of the geographical region do not affect the detection system. Additionally it enables the detection of all kinds of mines without regards to being plastic or metal. Because the speed at which the detection vehicle issues an alert as the result of analyzing the air takes around two percent of a second, a very rapid detection operation is performed. By virtue of this it is extremely useful especially for the mine scanning and detection operations of mobile military units.

Nitrogen is the building matter of all living cells. Nitrogen, which is especially vital for plants, is an element which accelerates growth and development in plants, and increases the amount of fiber, fruits and seeds. In the case of the soil being deficient in nitrogen, plants have a yellowish green color. It has been determined as the result of of research conducted that the nitrogen retained by plants is in organic form. Nitrogen which is found tied up in inorganic form in most soils, is in the form of ammonium (NH4+) and nitrate (N03*). Even though the amount of nitrite (N02) can sometimes be measured, when compared with ammonium and nitrate, it is seen that its quantity is very small and general accepted not to be adequate for detection. Despite this, it has been found that, in alkaline soils being subject to ammonium fertilization in a heavy manner, there is an accumulation of nitrite. Recent studies have shown that the nitrite oxidation by Nitrobacter is prevented by ammonium. This situation gives rise to the opinion that nitrite accumulation is the result of a high dose of addition of fertilizers containing ammonium or producing ammonium to soil which has an alkaline reaction. It is accepted that other inorganic nitrogen forms such as hydroxylamine, hypo-nitrous acid and imido-nitric acid (nitramide) arises as a byproduct during the microbial processes (nitrification, denitrification, N2 fixation etc.) which give rise to nitrogen change in the soil, that most of these compounds are not stable and therefore, they cannot be identified in soil. The hydroxylamine in mineral soils readily decomposes through a non-biological reaction and nitrogen in gaseous form is produced.

Until recently it was accepted that only a portion (2%) of total nitrogen was in inorganic forms. However, today, it is known that many soils have the property of fixing ammonium (non-alterable ammonium adsorbtion). Studied conducted recently have revealed that certain soils contain a significant amount of fixed ammonium and that this ammonium is not detected via the methods used for the detection of inorganic nitrogen forms in soils. According to existing information, the part of the soil's nitrogen, which is not alterable generally does not exceed 5% in surface soils. However, this amount may exceed 30% in certain subsoils. The mechanism by which ammonium is held by the soil is not yet fully explained. Despite this, it is know that the organic and inorganic portions of the soil have the capability of fixing ammonium and that the fixed ammonium is reported to be in grids of silicate minerals. However, no definite evidence exist that all of this fixed ammonium is related to silicate minerals.

It is known that many soils have the capability of holding ammonium in a form which is not extractable by methods used in extracting cations. Some soils contain significant measures of fixed ammonium and therefore, when the ammonium in soil is made mention of, it is needed that the terms “fixed” and “alterable” are identified. Unfortunately no descriptions on which consensus exist are available for these terms.

According to the Soil Science Society of America, ammonium fixation is the adsorbtion of ammonium ions by “soil or minerals” in a manner not soluble in water or readily alterable. Despite this, ammonium and other cations, which are fixed by the soil and clay minerals may be extracted by cation exchange processes, depending on the nature of the cation used, and the amount extracted by the other cation (potassium, rubidium, cesium) which can be fixed, is much less that that extracted by a cation which cannot be fixed (sodium, calcium). That the small amount of K found in the solution blocks the release of fixed ammonium, has again been determined by many researchers.

In the light of such inventions obtained, ammonium fixation is described as “ammonium adsorbed in a manner as not to be capable of being replaced with K+”. However, this description is also not sufficient, because the amount of ammonium released by K+ from soils and minerals containing fixed ammonium, depends on the type of the K+ solution used and the conditions. Accordingly, even if K+ used as the displacing cation, the properties of the detection method need to be specified in the description of the fixed ammonium. For practical purposes many researchers have argued that it is sufficient to describe fixed ammonium as “ammonium which cannot be extracted with I N KC1 at laboratory temperature”. If this description is adopted, it would seem fitting that alterable ammonium is also determined as “ammonium which can be extracted with this solution”. It has been agreed that it is necessary to distinguish between the ammonium fixed after the addition of ammonium to the soil or to minerals and the fixed ammonium present in these materials before the addition of ammonium, and the term “natural” has been used for this latter form. Despite this, this term gives rise to an inaccurate meaning that the fixed ammonium arisinf naturally in soils and minerals has occurred during the creation of these materials and not in recently.

In the light of these facts, it would be possible to say that a mine found buried under the soil intensively released nitrogen in inorganic form into the environment. The invention titled “Method and device for land mine detection by measurement of nitrogen gas”, on the other hand, is an innovation which enables the mine detection through the measurement of the nitrogen in question. In an environment, where the amount of inorganic nitrogen found in free form in nature does not exceed 2% without the presence of an external factor, the analysis of the air in the environment via sensors and the device issuing an alert in the event the amount of nitrogen measured exceeds this ratio, comprises the basis of the invention.

### DESCRIPTION OF FIGURE

FIG. 1: Appearance of the land mine detection device

### DESCRIPTION OF PARTS

1. Sensor point

2. Air analysis sensor

3. Conductive cable

4. Processor unit (not shown in FIGURE)

5. Processor indicator panel

6. Processor keypad

7. Warning light

8. Processor unit pocket clamp

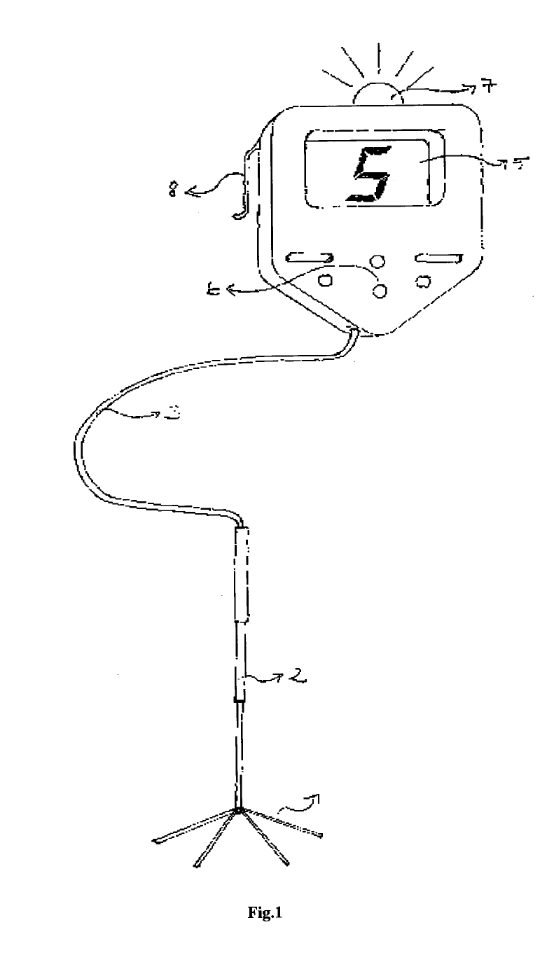

The invention relates to the method and device for land mine detection by measurement of nitrogen gas (FIG. 1). The land mine detection device (FIG. 1) comprises 8 different components. These components are respectively, the sensor point (1), the air analysis sensor (2), conductive cable (3), processor unit (4), processor indicator panel (5), processor keypad (6), warning light (7), and the processor unit pocket clamp (8). The air in the environment is sent to the air analysis sensor (2) via the sensor points (1). Via the sensors located inside the air analysis pipe the amount of inorganic nitrogen in the air is measured and their data are sent to the processor unit (4) via the conduction cable (3). The processor unit (4) evaluates the incoming data via a simple piece of software and compares it to the ratio of previously completed nitrogen ratio. If the processor unit (4), which displays, via the indicator panel (5), the data found as the result of the measurement, encounters any measurement which is higher than the value defined, it warns the operator by turning on the warning light (6). The warning is performed both by the lighting of the warning light (6) and by the high vibrating battery, to which the unit is connected, switching to the vibration mode. To facilitate the operator's use, the operator has the possibility to hang the processor unit (4) via the processor unit pocket clamp (8) as desired. The keypad located on the processor unit (4) is called the processor keypad (8). The processor keypad (8) is the interface which provides input for the purpose of transferring external data to the processor unit (4). For example, data such as the turning on and off of the warning modes, the adjustment of the warning intensity and the sensitivity of comparison of the processor unit (4), are entered into the system via the keys located on the processor keypad (8).

1. The invention relates to a land mine detection device, characterized in that; it comprises the air analysis sensor (2), which measures the air in the environment in which it is located and determines the amount of inorganic nitrogen found in the environment, and the processor unit (4) which controls the system.

2. The land mine detection device according to claim 1, characterized in that; it comprises the sensor points (1) connected to the air analysis sensor (2) through which air is sucked in and the measurement is conducted.

3. The land mine detection device according to claim 1, characterized in that; it comprises the conductor cable (3) which transmits the analysis data to the processor unit.

4. The processor unit (4) according to claim 1, characterized in that; it is the processor unit (4) which evaluates the received data via the software and compares with the previously defined nitrogen ratio and reflects the data obtained to the indicator panel and operates the warning units such as the warning light and vibration and interprets the data entered into the system by the operator.

5. The land mine detection device according to claim 1, characterized in that; it comprises the processor keypad (6) which enables data to be entered into the system by the operator.

6. The land mine detection device according to claim 1, characterized in that; it comprises the processor indicator panel (5) which performs the display of all kinds of information for the operator.

7. The land mine detection device according to claim 1, characterized in that; it comprises the warning light (7) designed to warn the operator in the event of a measurement higher than the limit value.

8. The land mine detection device according to claim 1, characterized in that; it comprises the clamp (8) which enabled the processor unit to be hanged.

9. The invention relates to land mine detection method by measurement of nitrogen gas, characterized in that; it comprises the process steps of:
measurement of the amount of nitrogen in the environment via the sensors (2) located on the device,
transmission of the data obtained to the processor unit via the conductor cable,
the detection of the mine in the event the measurement value exceeds the previously detected critical level.

10. The land mine detection method according to claim 9, characterized in that; it can enter into the system the threshold value for the amount of inorganic nitrogen used in the analysis.

11. The land mine detection method according to claim 9, characterized in that; in the event the data obtained as the result of the analysis exceeds the previously defined threshold value, it warns the operator via the warning light and the high vibration battery.

12. The land mine detection method according to claim 9, characterized in that; it presents the data obtained as the result of the analysis and the instantaneous information on the system to the operator via the indicator panel.
